(19) United States
(12) Patent Application Publication
(10) Pub. No.: US 2020/0094761 A1
(43) Pub. Date: Mar. 26, 2020

(54) Seat occupation, vital signs and safety belt lock sensor system for rear vehicle seats without power supply

(71) Applicant: NOVELIC D.O.O., Belgrade (RS)

(72) Inventors: Darko TASOVAC, Belgrade (RS); Veljko MIHAJLOVIC, Belgrade (RS); Veselin BRANKOVIC, Belgrade (RS)

(73) Assignee: NOVELIC D.O.O., Belgrade (RS)

(21) Appl. No.: 16/140,319

(22) Filed: Sep. 24, 2018

(51) Int. Cl. B60R 21/015; B60N 2/00

(52) U.S. Cl. CPC B60R 21/0153; A61B 5/0205; B60R 21/01546; B60N 2/002

(57) ABSTRACT

The present invention discloses seat occupation, vital signs and safety belt lock sensor for rear seats, which do not have power supply. The proposed system contains mm-wave radar sensor to be used for rear seat detection of seat occupancy and for vital sign detection, being placed on the vehicle ceiling or being integrated in the front seats, having radiation in the direction of the rear seat and detection if the human being is on the rear seat. The proposed system further contains hardware functionality being integrated in the safety belt environment. This functionality is communicating status if the safety belt is locked or not, in the case when the remote mm-wave sensor detected the human being on the rear seat, using arbitrary wireless communication means, and embedded arbitrary means for conserving energy, like battery. The proposed system is additionally detecting vital signs of the person using rear seats.

### TECHNICAL FIELD

The present invention relates to a sensor system and method of operation addressing rear seats occupancy and vital sign detection of the humans, in the vehicles, using radar-based sensors in mm-wave frequency ranges, when the rear seats do not have power supply.

### BACKGROUND ART

The specific problems or tightly attached, at least two, rear seats close to each other without power supply are addressed. The rear seats are in the vehicle or on the passenger's sides in busses, and in trains. Especially the case of three rear seats in personal vehicles is addressed.

In many practical applications the rear seats in the personal car or in busses or trains do not have electricity, due to increased system cost of the vehicles and due to the demand to have movable, or fast replaceable low-cost rear seats.

The problem is to introduce the innovative solution reducing the system cost, by providing seat occupancy and vital signs detection of humans on the seats, without electricity and cabling in the seats. The Governments impose that vehicles must have control of the rear seat belts usage in the case of the seat occupancy. State of the art technology imposes to use power supply in the rear seats, required for the pressure sensors in the seat and for the locking belt switching sensor. These increases the system cost of the vehicle and rear seats handling, as well as movement flexibility due to vehicle power support cables.

There is a strong motivation to deploy new generation of the sensors for the following application scenarios of the rear vehicle seats without power supply:
a) Detection one or more of a human being on the rear seats in one row.
b) Vital signs detection of a one or more human being on the rear seats in one row.
c) Having information about seat occupancy, to check if the seat belt on specific detected occupied seat is fasten
d) To communicate to the vehicle system, if the safety belt of the rear seat, switched the status from un-locked to the locked

The related system and method of operation should be able to address all four outlined application scenarios.

State-of-the-art radar-based sensors are addressing seat occupation, and vital signs acquisition problem, without addressing system cost minimisation of the complete vehicle seat row monitoring with associated safety belts usage monitoring, when the rear seat does not have power supply.

Proposed system in this invention and method of operation, are solving this problem.

The following patents and patent applications show the relevance of the topic and the state-of-the-art sensor sensors addresse seat occupancy detection.

U.S. Pat. No. 9,865,150, “Millimetre-wave seat occupation radar sensor”, discloses the mm-wave seat occupation sensor, being related to one seat observation. This invention do not address the seat belt connection, and rear seat problem.

US20160311388, “Radar sensor with frequency dependent beam steering”, discloses a radar system with frequency scanning for in-vehicle occupant detection and classification for seat-belt reminder functionality (SBR) and airbag suppression. The system includes an antenna system for which the main lobe direction (azimuth angle ö) is changing mostly linearly over frequency. This system requires complicated and large size mm-wave antennas, which in the same time do not allow sensor, the fine adjustment of the sensor for different type of the vehicle, where the angular positions of the beams would need to be different for different vehicle types, which impose the complete redesign of the antenna systems. This system does not address the problem of the possible rear seat constellation without power supply.

U.S. Pat. No. 6,753,780, “Vehicle occupant detection system and method using radar motion sensor” discloses A motion sensing system and method for detecting an occupant in a vehicle by movement. This system does not address the problem of the possible rear seat constellation, addressing seats being closed, neither application problem with seats without power supply.

U.S. Pat. No. 6,026,340, “Automotive occupant sensor system and method of operation by sensor fusion” discloses system for sensing the presence, position and type classification of an occupant in a passenger seat of a vehicle. In a preferred embodiment, infrared sensor inputs and ultrasonic sensor are used instead of radar, which bring system and cost disadvantage compared to the proposed solution.

WO2015127193A1, “Vehicle seat with integrated sensors”, addresses electronics for vital signs detection being integrated in the seat. The approach of the sensor being integrated in the seats bring system disadvantages.

U.S. Pat. No. 8,725,311, “Driver health and fatigue monitoring system and method”, addresses Vehicle including a seat in which an occupant sits during use of the vehicle and a monitoring system for monitoring the occupant in the seat. The approach of the sensor being integrated in the seats bring system disadvantages.

U.S. Pat. No. 6,362,734, “Method and apparatus for monitoring seat belt use of rear seat passengers” discloses a system for monitoring the secured/unsecured status of seat belts in rear seating rows of a vehicle notes the number and/or location of seat belts secured at a base time when all doors are closed and the vehicle starts in motion, and imposes that the other sensor being integrated in the seat is checking occupancy.

In the following patents and patent further background of the problems and similar applications are addressed:

U.S. Pat. No. 6,445,988B1, “System for determining the occupancy state of a seat in a vehicle and controlling a component based thereon”

U.S. Pat. No. 6,026,340A, “Automotive occupant sensor system and method of operation by sensor fusion”

DE10259522A1, “Radar-based sensing of position and/or movement of the body or in the body of living beings”

DE502004002969D1, “Seat occupancy sensor”

DE10200703217154, “Device for detecting the occupancy state of a vehicle seat”

JP2011063076A, “Seating sensor for vehicle”

WO2012053620A1, “Seat device and method for disposing seat occupancy sensor used therein”

US20120242492A1, “Seat occupancy detection and display system”

### SUMMARY OF INVENTION

This invention proposes apparatus with part 100 and part 2000, as well as the method of operation for occupation and safety belt sensor, being able:
To detect seat occupation of the rear seats
To detect optionally vital signs of the persons on the rear seats
To detect if the safety belts at the rear seats, where the persons are detected by the same apparatus are fasten
To have wired or wireless interface to the entity to transmit information about detected events, like occupation and fasten seat belts to the vehicle infrastructure
To have optional ability to initiate the specific actions upon detection of the specified pre-event events
To be optionally integrated in more complex vehicle sub-system like front seats
To be optionally integrated in more complex vehicle sub-system like ceiling light system for rear sets,

Where the rear seats of the vehicle do not have power supply.

The proposed system has two apparatuses: apparatus 100 and apparatus 2000, providing pre-requisites for the associated proposed method of operation.

The applications scenarios for positioning apparatus 100 are shown in the FIG. 21 and FIG. 2a for the apparatus 100 being placed in the vehicle environment above the rear seats and not in one of the front seats, and in FIG. 1b and FIG. 2b for the apparatus 100 being placed in one of the front seats.

The key system-relevant components of the proposed apparatus 100 are:
High-gain planar antenna system, realized by the plurality of the technologies, with at least one receiving antenna system 110, 120, 130, 140 and at least one the transmit antenna systems 21 and 22 each of them having more than one antenna radiation elements, and operation in the mm-wave frequency band.
Millimetre-wave radar with integrated front end on silicon 10, system on chip, providing analog processing of the mm-wave signal, and the provision of the analog to digital conversion functionality;
Digital signal processing functionality 30
Mechanical assembly with power supply interface to power supply infrastructure in the vehicle or front seats, containing mechanically integrated antenna, digital and analog functionalities and having mechanical connection to the vehicle or seat infrastructure
Supporting circuitry 50 as a part of apparatus 100 may include functionalities like light warning source, by the plurality of the realization options
Interface sub-system 60 allowing connection to the vehicle infrastructure 1000.
Wireless entity 400, containing communication systems, being realised by the plurality of the wireless communication means protocols, preferably with low power communication system like low power Bluetooth, operation in ISM frequency band.

The applications scenarios for apparatus 2000 are shown in the FIG. 3. The apparatus 2000 is integrated in the safety belt 3000 of the rear set.

The key system-relevant components of the proposed apparatus 2000 are:
Wireless entity 401, containing communication systems, being realised by the plurality of the wireless communication means protocols, preferably with low power communication system like low power Bluetooth, operation in ISM frequency band
Battery 403, representing power source for entity 401, 402 and 403.
Switch sensor 402, detecting if the safety belt 3000 is locked or not locked
Light source 404, preferably colour diode, which is indicating if the Battery 403, needs to be replaced, in the case of not having enough power for the operation.

The proposed apparatus and method of operation allow and facilitate production of the complete sensor systems being low cost, and reducing the system cost of the vehicle, enabling safety functions on the rear seats, without power supply in the rear seats.

The choice to use the mm-wave frequency band (30 GHz to 300 GHz) and advantageously to use non-licenced 60 GHz band, ISM 60 GHz Band and 79 GHz Automotive band, is mainly related to the size of the antenna system allowing very small and compact device, even though it contains the high-gain antenna with more than one radiation elements.

Following operation steps, being part of the proposed method of operation are executed:
All rear seats are checked if the person is seating on one of the rear seats, by using apparatuses 100, and method of the operation being defined as “human being detection method”.
By using apparatus 100 and apparatus 200, and method of the operation being defined as “method for safety belt locked detection and information communication method”, information if the safety belt is locked on the rear seat position, where previously the person was detected by “human being detection method”, is communicated to the apparatus 100.
Apparatus 100 is sending information, which seats are occupied and if the related safety belt is locked, by the plurality of the wireless and wired means to the vehicle infrastructure.
Apparatus 100 is optionally calculating respiratory frequency of person being detected on the dedicated rear seat, and it is sending this information by the plurality of the wireless and wired means to the vehicle infrastructure.
Apparatus 100 is optionally calculating heart beat frequency of the person being detected on the dedicated rear seat, and it is sending this information by the plurality of the wireless and wired means to the vehicle infrastructure.

### BRIEF DESCRIPTION OF DRAWINGS

FIG. 1a-1b present apparatus typical application scenarios, with top vehicle view, where:

FIG. 1a where the rears seats do not have power supply, and apparatus 100 is placed in the vehicle environment, outside the front seats, facing the rear seats with the specific high gain antenna pattern.

FIG. 1b where the rears seats do not have power supply, and apparatus 100 is placed in the vehicle environment, inside the front seats, facing the rear seats with the specific high gain antenna pattern.

FIG. 2a-2b presents apparatus typical application scenarios, with lateral vehicle view, where:

FIG. 2a where the rears seats do not have power supply, and apparatus 100 is placed in the vehicle environment, outside the front seats, facing the rear seats with the specific high gain antenna pattern.

FIG. 2b where the rears seats do not have power supply, and apparatus 100 is placed in the vehicle environment, inside the front seats, facing the rear seats with the specific high gain antenna pattern.

FIG. 3 presents the rear seat environment without power supply, containing, apparatus 100 for person occupancy detection and apparatus 2000, being integrated in the seat belt system 3000, where apparatus 2000 is communicating with the apparatus 100 with wireless means, and where apparatus 100 is using radar principle detecting occupancy of the seats, and optionally vital signs of the person on the seat.

FIG. 4 presents the apparatus 100 functional hardware blocks, and two types of the wireless activity, radar based in mm-wave frequency bands, and low power communication means in ISM frequency band.

FIG. 5 presents the apparatus 2000 functional hardware blocks, and two wireless activity, being realized with low power communication means in ISM frequency band.

FIG. 6 presents possible antenna arrangement for Apparatus 100, where the Apparatus 100, has digital beam forming functions and addresses up to four seats

FIG. 7a-7b presents possible antenna arrangement for Apparatus 100, where the Apparatus 100, does not have digital beam forming functions

FIG. 7a where Apparatus 100 can address only one rear seats, with one Rx antenna 110 and one Tx antenna 21, where complete antenna system is realized by the planar feeding means in one plane

FIG. 7b where Apparatus 100 can address three rear seats, with three Rx antennas 110, 120 and 130 and one Tx antenna 21, where complete antenna system is realized by the planar feeding means in one plane

FIG. 8 presents possible Apparatus 100, realization option, where FIG. 7b is implemented.

### DESCRIPTION OF EMBODIMENTS

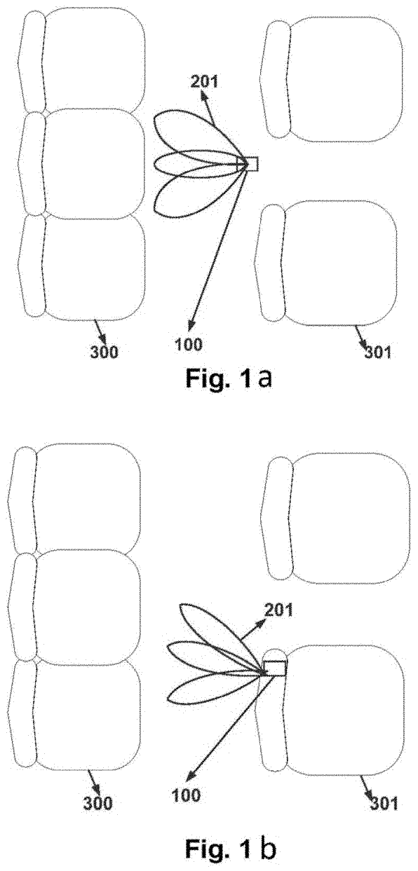

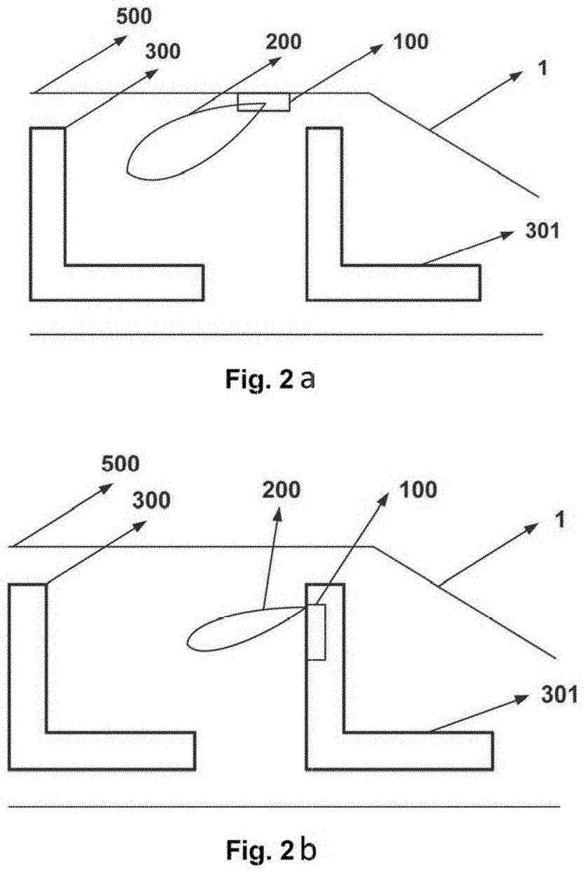

The proposed system contains two HW parts: first apparatus 100 and second apparatus 2000. The first apparatus 100 with mm-wave HW radar functionality, being placed inside of the cabin, facing rear seat under observation and apparatus 2000 being integrated to the safety belt portion of the rear seat. MM-wave radar operation comprises operation in the between 30 and 300 GHz. Advantageously automotive frequency band 77-81 GHz, and non-licensed 60 GHz bands are used, providing small antenna sizes for high gain radiation mode. The proposed first apparatus 100 has at least one high gain receive antenna and at least one high gain transmit antenna, where the antenna has minimum two antenna elements, to provide bundling of the radiation beam in the specific direction towards the specific rear seats, like seen in the FIG. 1a-1b and FIG. 2a-b. Detection of the seat occupation is calculated using analysis of the specific vital signs vibrations imposed by the human being. The narrow antenna beam is required to ensure that event of person detection is not influenced by the occupation by the neighboured seat which would lead to the false detection, as well as to false vital sign reading. That is why the antenna radiation beams 201 in the azimuth must be narrow as seen in the FIG. 1a-1b and FIG. 2a-2b. The rear seat in FIG. 1a-1b and FIG. 2a-2b. do not have power supply, to minimise the sensor system cost, and or to have an option to move the rear seats more easily with power supply, and or to move the rear seats out of vehicle or to exchange them easily. Imposed by the law regulation, and by the demand to increase the safety in the vehicle, it is necessary to detect if the safety belt of the rear seat is occupied by the human being is locked or not. This information needs to be known by the vehicle system, to ensure warning for using safety belts, or to get the information if in case of accidents specific vehicle systems like airbags should be enabled or not.

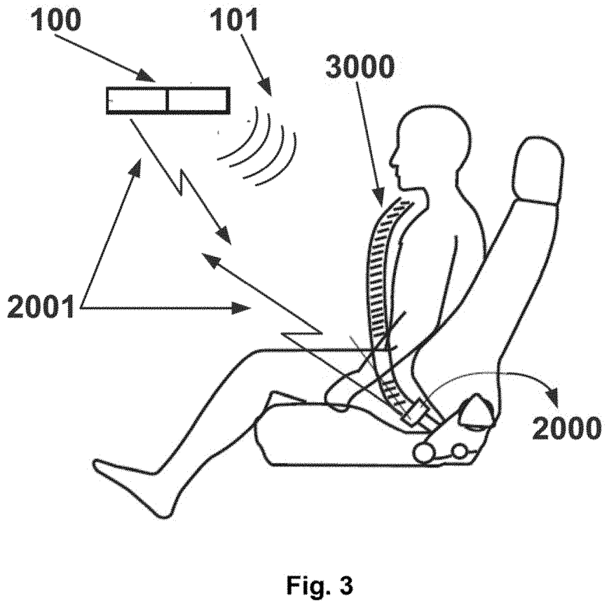

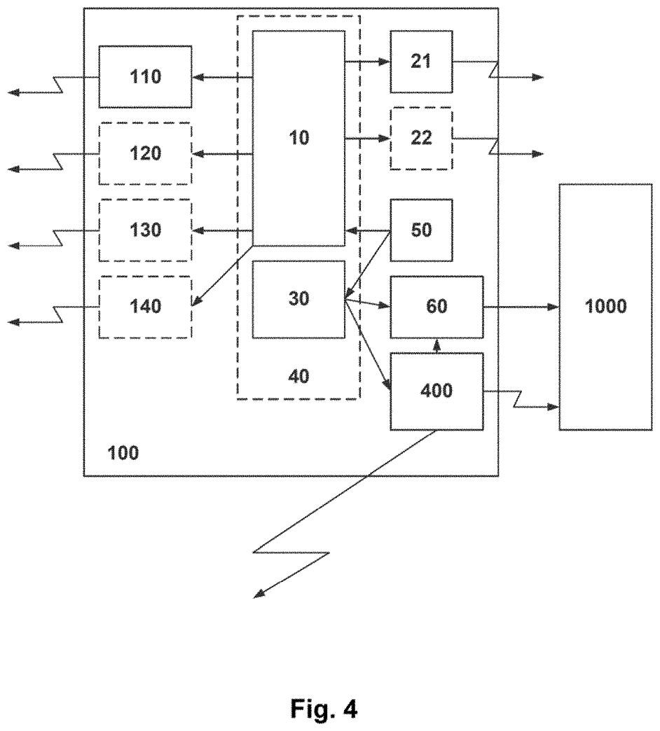

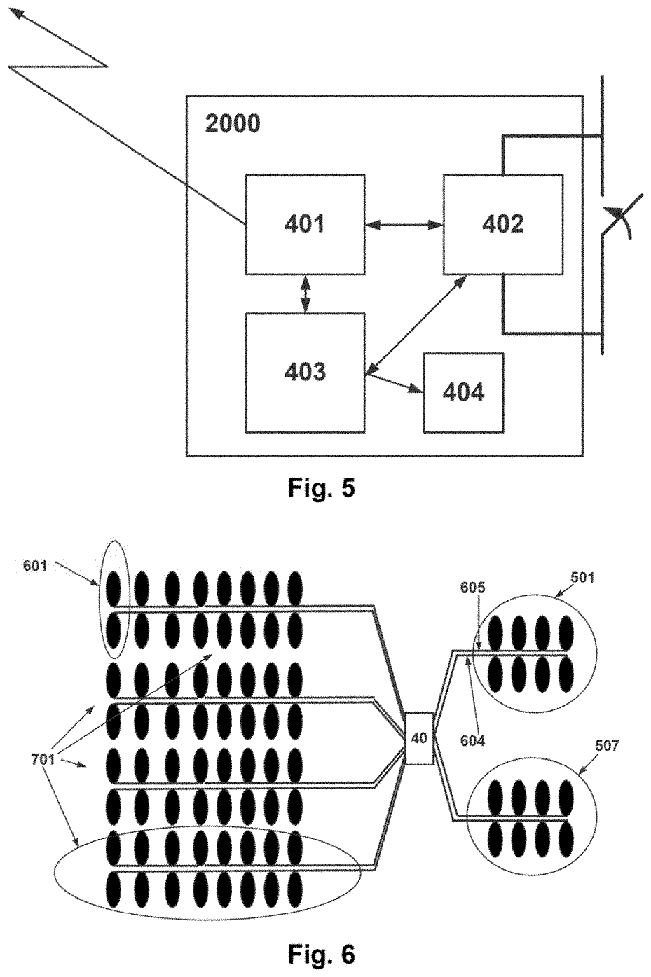

As seen in the FIG. 3 the proposed first apparatus 100 contains:
1. At least one high-gain planar antenna for transmitting mm-wave radio signals 21, where the high-gain planar antenna has at least two radiation elements;
2. At least one high-gain planar antenna for receiving mm-wave radio signals 110, where the high-gain planar antenna has at least two radiation elements;
3. Integrated mm-wave radio front end 10, implemented in arbitrary semiconductor technology, having on-chip integrated mm-wave voltage control oscillator with PLL, mm-wave power amplifier, at least one mm-wave IQ demodulator, digital control interface, power supply;
4. Digital processing functionality 30 with arbitrary hard wired and SW digital processing capability, being able to digitally process the signal coming out of the entity 10, including controlling functionality and calculation and memory capacity for performing digital signal processing by arbitrary type of the realization options
5. Wired communication interface 60 to connect first Apparatus 100 to the vehicle infrastructure entity 1000, being outside the apparatus 100, being released by the plurality of the technologies and communication protocols
6. Supporting circuitry 50, including mechanical interface to vehicle environment 1000, where the first Apparatus 100 is connected to the vehicle environment, and supporting electronic circuitry for providing the power supply from the vehicle environment 1000 to the first apparatus 100.
7. Wireless communication entity 400, being able to establish wireless data communication between first apparatus 100 and second apparatus 2000, by using arbitrary non-licenced wireless communication means in frequency band lower that mm-wave frequency band, comprising at least one integrated antenna.

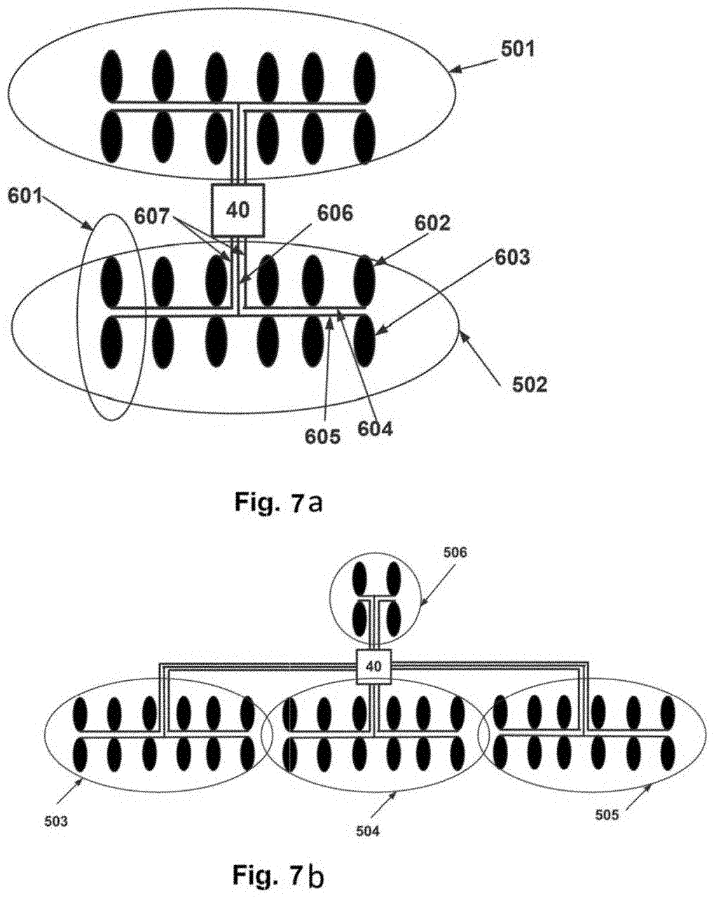

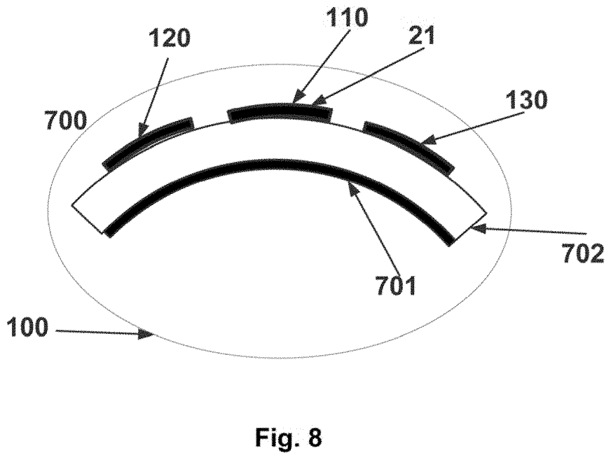

In the praxis, realistic practical vehicle application scenario-imposed cases, where two seats are to be observed, three seats are to be observed or two by two, in case of busses for example. The focused narrow radiation beams 200, 201 are required for receiving chains. That means the Apparatus 100 wold need to have one of the following options to generate one to four different high gain beams in the specific azimuth directions: to use for each direction high gain antennas with different special orientation like in FIG. 7a-7b, or to use set of the high gain antennas with digital or analog beamforming, like in FIG. 6, for example. If the transmit radiation is performed in wide radiation manner by transmit antenna 21 for example, beams are switched to specific seat to get the reflected signals, which further on need to be evaluated by the signal processing in the entity 30. If the beamforming is used, the ability for special forming is influenced by the number of the receiving chains and receiving antennas as well as with the number of the transmit changes. If the number is receiving chains limited the azimuth related beam switching capability is limited. That means that for each vehicle the position of the Apparatus 100 is influenced, by the limitation of the beam forming, due to fixed angles to be addressed. That means if the position of the Apparatus 100 in the vehicle is fixed by the mounting, like electricity connection close to inside cabin light apparatus above passenger, the rear seats would need to be irradiated by angles, which are not fixed as in case with beam forming with limited number of receiving and transmitter chains. In that case solutions like bended antenna systems in FIG. 8 by the antenna system of the FIG. 7a-7b may be advantageously used.

The second apparatus 2000 is placed inside of the safety belt system 3000, like seen in the FIG. 3. The apparatus 2000 is communicating with the apparatus 100 with wireless means. The distance between apparatus 2000 and apparatus 100 is chosen to be less than 3 meters to ensure on one side, high probability of the seat occupation detection by apparatus 100, denoted as radar sensing distance 101 in the FIG. 3. and on the other side ultra-low power wireless communication between apparatus 2000 and 100, denoted by numbers 2001, on the FIG. 3.

The apparatus 2000 contains:
1. Wireless communication entity 401, being able to establish wireless data communication between second apparatus 2000 and first apparatus 100, by the same wireless communication means like entity 400, from first apparatus 100, comprising at least one integrated antenna
2. Entity 403, battery conserving electrical power, by the plurality of the realization options, representing power source for operation of the entities 401, 402 and 404.
3. Entity 402, being the switch sensor, detecting if the safety belt 3000 is locked or not locked
4. Entity 404, being the light source, which is indicating if the battery 403, needs to be replaced, due to inability to provide enough power for the operation of the entities 401, 402 and 404.

The low-power wireless means used by entity 401, can be: Low Power Bluetooth, UWB based low-power communication system or other ISM and non-licensed communication system, whereby the systems with power consumption below 1 mW are proposed, to ensure in specific low duty circle operation. This would enable to use commercially available battery source for one seat belt systems for the duration of several years.

Method of operation is proposed, utilizing the System being described. It contains two operation steps: “human being detection method”-operation step, being declared as first operation step, and “method for safety belt locked detection and information communication method”-operation step being declared as second operation step.

The first operation step has following sub-set of operations:
Transmission of mm-wave signals generated in 10 using 21;
Receiving mm-wave signals reflected from observation area using 110;
Digital processing of the signal in 30, by trying to detect at least one the human being vital signal patterns, by the plurality of the signal processing algorithm approaches
Decision event of: human being detected, or human being not detected is executed, by the plurality of the evaluation procedures of at least one of the human being vital signal patterns, being search for in the previous sub-operation
Information of the event detection is communicated to the vehicle environment 1000, by means of entity 60.

The second operation step being executed after first operation step, has following sub-set of operations:
initialisation of the second operation steps by the apparatus 100 and its entity 400, by sending information to start operation step two, to the entity 401, only in the case that event detection, of human being is detected, from first operation step is positive
checking by the entity 402 if the safety belt is locked
sending information from entity 402 to entity 401 about safety belt locked or not
sending information from entity 401 to entity 400 by wireless means, if the safety belt is locked
sending information from entity 401 to entity 400 by wireless means, if the safety belt changed status from locked to the unlocked
apparatus 100 is taking information from its entity 400 and over entity 60 is informing vehicle infrastructure, if the human being, being detected on the rear seats has safety belt locked, and if the status of safety belt changed from locked to the non-locked.

Besides the feature of apparatus 100 to detect the occupation of the seat by the human being, the apparatus 100, can be optionally used for detection of the respiratory frequency value, and heart beat value by utilisation of arbitrary signal processing activities. The vital sign information can be than stored for the vital sign profiling of the passengers, which may provide additional information to the vehicle system, also related to event calculation of stress conditions, emotion status and fatigue.

In the FIG. 6. transmit antennas 21, 22, corresponding to the planar antenna structures 501 and 507 as well as receiving antennas 110, 120, 130, 140, corresponding to the planar antenna structures of shape 701, all of them parts of the apparatus 100, are realized each by more than two dipole-based structures 601. Dipoles are realized by metalized surfaces in the same plane, and advantageously do not need to be realised as state of art patch antennas in mm-wave radar system, which require microstrip line for feeding and substrate with specific thickness. Proposed topology and realization with dipoles enable low-cost realization of the analog HW, without expensive substrates for patch antenna approach. The antenna parts half dipoles 602 and half dipoles 603 are fed by the differential coplanar line 604 and 603 feeding structure, also being realized on the same metalized surface as receiving and transmitting antenna parts. The radiation parts are half dipoles have arbitrary planar shape, where the maximum thickness of the one planar antenna shape is larger than one ⅕ of the wave length of the center frequency of operation. They can be realised as ellipsoid structures, or n-tagonal structures, N being larger than 5, with optional cut of surfaces at the end of surface to enable smaller sizes. The dipole parts are intentionally thick, to provide wide operation range, being larger than 20% for the center frequency of operation. This provides high yield, due to robust production tolerances allowed in the manufacturing process. The distances of the different dipoles 601 being fed by the same coplanar lines 604 and 605 are generally not the same to ensure proper radiation diagram and good matching of the antenna structure. Antenna dipoles are fed with differential type of feeding being suitable for differential type of the mm-wave generation in the entity 10. In the FIG. 7a-7b antenna parts are fed also by the coplanar line 604 and 605 differential feeding structure. However, coplanar lines 606 and 607 are introduced, working in current-operation mode. This enables single ended mm-wave feeding of the entity 10.

FIG. 7a-7b shows presents possible antenna arrangement for Apparatus 100, where the Apparatus 100, does not have digital beamforming functions. In FIG. 7a apparatus 100 can address only one rear seat, with one Rx antenna 501 and one Tx antenna 502, where complete antenna system is realized by the planar feeding means in one plane, and there is no possibility of the beam forming. In FIG. 7b Apparatus 100 can address three rear seats, with three Rx antennas 503, 504 and 505 and one Tx antenna 502, where complete antenna system is realized by the planar feeding means in one plane, and there is no beam forming. The transmit antenna 502 is advantageously released with wider beam, compared to 503, 504, and 505 antennas. In the FIG. 8 possible realization option is outlined using structures of the FIG. 7b to address specific radiation angle, being arranged according to the application scenario environment. Combination of the distances of the receiving antennas with bending angles of the structure may allow realisation of almost arbitrary angles, in contrast to the beam forming option. In FIG. 8 antenna structures are printed on thin dielectric material and them they are bended over specific foam 702, with low permittivity along one quarter of the frequency under operation, where the foam has on it opposite metallization or metalized plastic 703, serving as a reflector for the proposed antenna structure.

1: mm-Wave System comprising the first apparatus 100 with mm-wave HW radar functionality, being placed inside of the cabin, facing rear seat under observation and apparatus 2000 being integrated to the safety belt portion of the rear seat, where mm-wave declares operation between 30 and 300 GHz, and comprising the second apparatus 2000, where the seat with second apparatus 2000 does not have power supply coming out of the vehicle infrastructure,
where first apparatus 100 contains:
At least one high-gain planar antenna for transmitting mm-wave radio signals 21, where the high-gain planar antenna has at least two radiation elements;
At least one high-gain planar antenna for receiving mm-wave radio signals 110, where the high-gain planar antenna has at least two radiation elements;
Integrated mm-wave radio front end 10, implemented in arbitrary semiconductor technology, having on-chip integrated mm-wave voltage control oscillator, mm-wave power amplifier, at least one mm-wave IQ demodulator, digital control interface, power supply;
Digital processing functionality 30 with arbitrary hard wired and SW digital processing capability, being able to digitally process the signal coming out of the entity 10, including controlling functionality and calculation and memory capacity for performing digital signal processing by arbitrary type of the realization options
Wired communication interface 60 to connect first Apparatus 100 to the vehicle infrastructure entity 1000, being outside the apparatus 100, being released by the plurality of the technologies and communication protocols
Supporting circuitry 50, including mechanical interface to vehicle environment 1000, where the first Apparatus 100 is connected to the vehicle environment, and supporting electronic circuitry for provide the power supply from the vehicle environment 1000 to the first apparatus 100.
Wireless communication entity 400, being able to establish wireless data communication between first apparatus 100 and second apparatus 2000, by using arbitrary non-licenced wireless communication means in frequency band lower that mm-wave frequency band, comprising at least one integrated antenna,
where the second apparatus 2000 is placed less than 3 meters away from first apparatus 100 contains:
Wireless communication entity 401, being able to establish wireless data communication between second apparatus 2000 and first apparatus 100, by the same wireless communication means like entity 400, from first apparatus 100, comprising at least one integrated antenna
Entity 403, battery conserving electrical power, by the plurality of the realisation options, representing power source for operation of the entities 401, 402 and 404.
Entity 402, being the switch sensor, detecting if the safety belt 3000 is locked or not locked
Entity 404, being the light source, which is indicating if the battery 403, needs to be replaced, due to inability to provide enough power for the operation of the entities 401, 402 and 404.

2: Method of operation, utilizing the System being described in claim 1 where method of operation comprising two operation steps: “human being detection method” being declared as first operation step, and “method for safety belt locked detection and information communication method” being declared as second operation step,
where the first operation step has following sub-set of operations:
Transmission of mm-wave signals generated in 10 using 21;
Receiving mm-wave signals reflected from observation area using 110;
Digital processing of the signal in 30, by trying to detect at least one the human being vital signal patterns, by the plurality of the algorithm approaches and plurality of the statistic evaluations
Decision event of: human being detected, or human being not detected is executed, by the plurality of the evaluation procedures of at least one of the human being vital signal patterns, being search for in the previous sub-operation
Information of the event detection is communicated to the vehicle environment 1000, by means of entity 60
where second operation step being executed after first operation step, has following sub-set of operations:
initialisation of the second operation steps by the apparatus 100 and its entity 400, by sending information to start operation step two, to the entity 401, only in the case that event detection, of human being is detected, from first operation step is positive
checking by the entity 402 if the safety belt is locked
sending information from entity 402 to entity 401 about safety belt locked or not
sending information from entity 401 to entity 400 by wireless means, if the safety belt changed status from locked to the unlocked
apparatus 100, is taking information from its entity 400 and over entity 60 is informing vehicle infrastructure, if the human being, being detected on the rear seats has safety belt locked, and if the status of safety belt changed from locked to the non-locked

3: System according to claim 1, and claim 2 where the first operation steps of the method of operation contains has following additional sub-sets of operations:
Detection of the respiratory frequency value, by utilisation of arbitrary signal processing activities and averaging approaches by the plurality of the observation time durations
Information of the respiratory frequency value is communicated to the vehicle environment 1000, by means of entity 60

4: System according to claim 1, and claim 2 where the first operation steps of the method of operation contains has following additional sub-sets of operations:
Detection of the heart beat frequency value, by utilisation of arbitrary signal processing activities and averaging approaches by the plurality of the observation time durations
Information of the heart beat frequency value is communicated to the vehicle environment 1000, by means of entity 60

5: System according to claim 1, and claim 2, where the transmit antennas 21, 22, corresponding to the planar antenna structures 501 and 503 as well as receiving antennas 110, 120, 130, 140, corresponding to the planar antenna structures of shape 701, all of them parts of the apparatus 100, are realized each by more than two dipole based structures 601, being realised by metalized surfaces in the same plane,
where the antenna parts are fed by the differential coplanar line 604 feeding structure, also being realized on the same metalized surfaces as receiving and transmitting antenna parts,
where the radiation parts are half dipoles have arbitrary planar shape, where the maximum thickness of the one planar antenna shape is larger than one ⅕ of the wave length of the center frequency of operation
where at least two distances of the different dipoles 601 being fed by the same coplanar lines 604 are not the same

6: System like in claim 5
where the antenna parts are fed by the coplanar line 604 differential feeding structure, where the transition from single end ed feeding coming from the mm-wave chip entity 10, is released by coplanar lines.

7: System like in previous claims
where the antenna parts being realized as printed structures on one surface, and realized on the surface, which is bended, in the way that the related radiation of the at least two receiving antennas 120 and 130 have maximum radiation in the specific direction, without the need to use beam forming approaches.

8: System according to previous claims, when the apparatus 100 is integrated in the vehicle seat 301

9: System according to claims 1-6, when the apparatus 100 is integrated in the vehicle ceiling 500
